(19) United States
(12) Patent Application Publication
(10) Pub. No.: US 2020/0001040 A1
(43) Pub. Date: Jan. 2, 2020

(54) METHOD, APPARATUS, AND SYSTEM FOR MEDITATION

(71) Applicant: Levels Products, Inc., Claremont, CA (US)

(72) Inventors: Peter B. Gloria, Claremont, CA (US); Himanshu Patel, Rancho Santa Margarita, CA (US)

(21) Appl. No.: 16/442,795

(22) Filed: Jun. 17, 2019

Related U.S. Application Data
(60) Provisional application No. 62/691,150, filed on Jun. 28, 2018.

(51) Int. Cl. A61M 21/02; G16H 20/70; A47C 7/02; A61B 5/0205

(52) U.S. Cl. CPC A61M 21/02; G16H 20/70; A61M 2021/0022; A61B 5/0205; A47C 7/021

(57) ABSTRACT

A method, apparatus, and system for meditation are described. The disclosed embodiments describe, amongst other things, a sitting apparatus that may monitor user biometrics. A software application for guiding the user through a meditation session, and the two used together as a system. The system may be included as stand alone sitting device, or the system may use a sitting apparatus and a second device. Also, described is a group mediation embodiment.

### I. CLAIM TO PRIORITY UNDER 35 U.S.C. § 119

The present application for patent claims the benefit of U.S. Provisional Application No. 62/691,150 filed on Jun. 28, 2018, entitled, “Contactless Sitting Device Monitor for Adaptive Breath and Calmness Training with Group Coherence,” assigned to the assignee hereof, and expressly incorporated herein by reference in its entirety.

### BACKGROUND

### I. Field

The disclosed embodiments relate to a method, apparatus, and system for meditation.

### II. Background

Meditation is a technique used to train one's mind. It may be considered a form of mindfulness that can improve an individual's mental, emotional, physical, and spiritual quality of life. However, sometimes it may be difficult to learn how to get into a state of meditation, stay in the state, optimize the state, or objectively track/monitor your history and experiences. Some existing meditation tools that you wear may be awkward to use and hinder rather than promote meditation. Therefore, there is a need in the art to have a comfortable, customizable, convenient, and objective way for helping people meditate.

### SUMMARY

In an embodiment, described is a sitting apparatus for meditation, comprising: a cushion; a sensor assembly module, the sensor assembly module comprising: a power module adapted to power the sensor assembly module; a sensor module adapted to obtain user biometrics; a signal conditioning module adapted to condition signals from the sensor module; a communication module; a processor module, the processor module adapted to send and receive information to a second device by utilizing the communication module, to send user biometric information to the second device meditation software application; and a memory module coupled to the processor module.

In yet another embodiment, described is a non-transitory computer readable medium encoded with processing instructions, for meditation software application, comprising: code to determine whether a user is new: if a user is new, then code to prompt a user to register and pair, if a user is not new, then code to automatically pair; code to calibrate a meditation session; code to allow a user to enter meditation settings; code to begin guided meditation; code to end guided meditation; and code to store the guided meditation session statistics.

In another embodiment, described is a system for mediation, comprising: means for a second device, the second device being capable of running instructions for a meditation software application; means for a sitting apparatus, the sitting apparatus being capable of use with the second device for meditation, comprising: means for cushioning; means for sensing user biometrics, the means for sensing user biometrics comprising: means for powering the sitting apparatus; means for obtaining user biometrics; means for signal conditioning; means for communicating, the means for communicating having means for communicating with the second device meditation software application; means for processing, the means for processing comprising means for sending and receiving information to the means for the second device; and means for storage.

### BRIEF DESCRIPTION OF THE DRAWINGS

The following embodiments may be better understood by referring to the following figures. The figures are presented for illustration purposes only, and may not be drawn to scale or show every feature, orientation, or detail of the embodiments. They are simplified to help one of skill in the art understand the embodiments readily, and should not be considered limiting.

FIG. 1A illustrates a sitting apparatus in an embodiment.

FIG. 1B illustrates a sitting apparatus in an embodiment.

FIG. 2 illustrates a sensor assembly module in an embodiment.

FIG. 3 illustrates a method for meditation in an embodiment.

FIG. 4 illustrates a guided mediation method in an embodiment.

FIG. 5A illustrates a method for the sitting apparatus in an embodiment.

FIG. 5B illustrates a method for the sitting apparatus in another embodiment.

FIG. 6 illustrates a system for mediation in an embodiment.

FIG. 7 illustrates a system in an embodiment.

FIG. 8 illustrates a group meditation system in an embodiment.

FIG. 9 illustrates code for meditation in an embodiment.

FIG. 10 illustrates code for guided meditation in an embodiment.

FIG. 11 illustrates a method for group mediation in an embodiment.

### DETAILED DESCRIPTION

Each of the additional features and teachings disclosed below can be utilized separately or in conjunction with other features and teachings to provide a method, apparatus, and system for meditation. Representative examples of the following embodiments will now be described in further detail with reference to the attached drawings. This detailed description is merely intended to teach a person of skill in the art details for practicing the preferred aspects of the teachings and is not intended to limit the scope of the embodiments.

The disclosed embodiments describe a meditation system comprising a sitting apparatus and a meditation software application, a sitting apparatus itself, and a meditation software application itself. The methods, apparatus, and systems may be used individually or as a group. The disclosed embodiments describe a sitting apparatus that comprises a variety of sensors and circuitry that may obtain, store, process, indicate, and transmit and receive information. For example, the information may include things like user biometrics, control signals, indications, alerts, and feedback. The sitting apparatus may send raw or processed information wirelessly, or wired, to a second device. In an embodiment, the sitting apparatus may send the information to be processed and displayed to a second device. The second device may have an application that processes, sorts, filters, analyzes, provides user feedback, sharing of data, data history, data storage, coordination of other users, and sending and receiving of information, including controls, from a sitting device. In an embodiment, the sitting apparatus may comprise a mediation software application and be fully capable of processing, filtering, analyzing, providing user feedback, sharing of data, storing of data, and coordination of other users (e.g. a stand alone device). In an embodiment, the sitting apparatus comprising a meditation software application may send information to be displayed to a second device (e.g. smart T.V., computer screen). Sitting apparatus, herein, broadly means anything that a user may want to sit on or have body contact with while in a meditation pose, but not necessarily wear, during meditation. For example, a cushioned seat, a padded mat, or pillow. A user may lean up against the sitting apparatus, sit on it, lay on it, or partially lay on it in order to make contact for use without wearing it. The second device may be any device capable of receiving information and transmitting information to a sitting apparatus. For example, the second device may be an iPad®, smart T.V., laptop, tablet, smart phone, an external server, computer, computer screen, a custom receiving device, cell phone, mobile phone, handheld radio, PDA, e-reader, personal wearable device, personal fitness device, digital home assistant, or any device capable of transmitting and receiving information wirelessly or wired and, when applicable, able to store, process, and display information. The meditation software application, amongst other things, may utilize user biometrics to analyze and provide user feedback during a meditation session. The meditation software application may provide “real time” guidance to users individually or as a group. The meditation software application may be running on a second device or on the sitting apparatus. The meditation software application may store, analyze, share, and display user statistics (e.g. historical data).

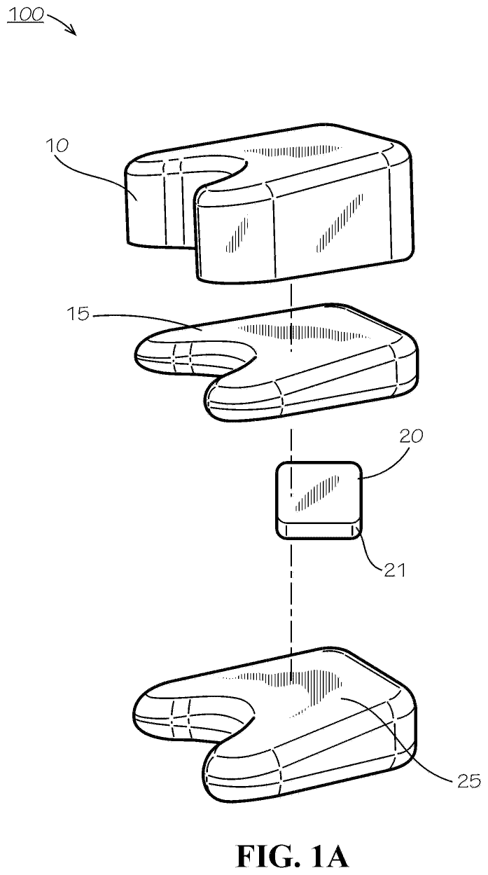

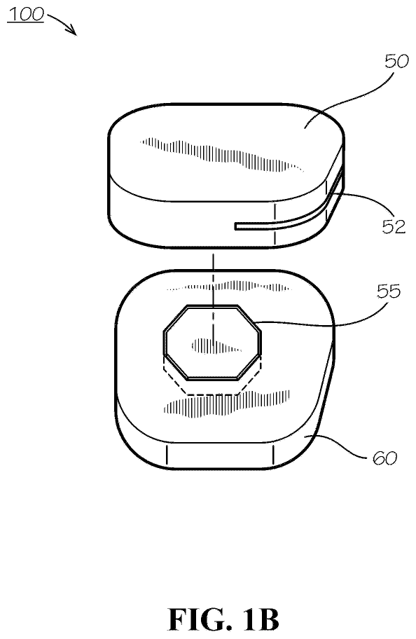

FIG. 1A illustrates a sitting apparatus 100 in an embodiment. In an embodiment, sitting apparatus 100 may comprise a cover 10, a first cushion 15, a sensor assembly 20, and a second cushion 25. Sensor assembly 20 may comprise an outer shell, enclosure, box, or casing 21 to house the internal components of the sensor assembly 20. In an embodiment, the housing 21 may be EMC shielded on the top, which may help to minimize EMI going through to a user. Sensor assembly (or sensor assembly module) 20 may be sandwiched in-between the first and second cushions 15 and 25. In an embodiment, the sensor assembly 20 may be embedded into an injection foam molded cushion 15. In an embodiment, as shown in FIG. 1B, there may be a single cushion 60 instead of two. The cushions 15, 25, 60 may comprise foam, padding, stuffing, pillow, memory foam, or any suitable material for sitting or laying on that provides a level of comfort for a user during meditation. Cover 10 may be any suitable material used for covering a sitting apparatus. For example, cover 10 may comprise cloth, fabric, padded fabric, rayon, vinyl, plastic, or any combinations thereof. Cover 10 may be removably secured in order to be washable and may comprise zippers, fasteners, strings, ties, buttons, or hook-loop fasteners as is well known in the art.

FIG. 1B illustrates a sitting apparatus 100 in an embodiment. In an embodiment, sitting apparatus 100 may comprise a cover 50, a cushion 60, a sensor assembly module 55 Sensor assembly module 55 may be inserted or embedded into cushion 60. Cover 50 may have a zipper or other equivalent functionality 52 of allowing the cover to be removably secured in order to be washed and placed back on separately from the cushion and sensor assembly.

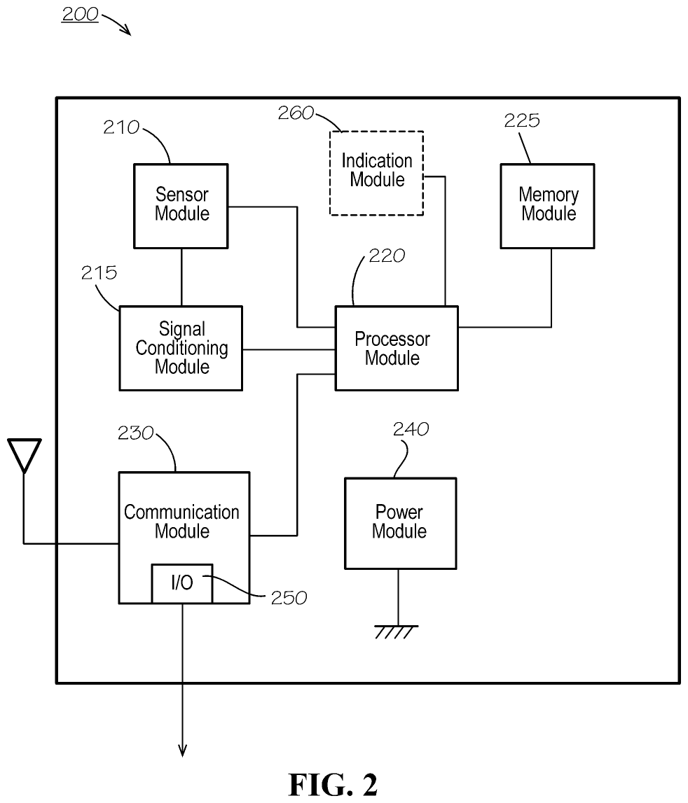

FIG. 2 illustrates a sensor assembly module 200 in an embodiment. Sensor assembly module 200 may have an outer housing. Sensor assembly module 200 may comprise a processor module 220, a memory module 225, a signal conditioning module 215, a sensor module 210, a communication module 230, and a power module 240.

In an embodiment, power module 240 may comprise an internal battery. Power module 240 may provide circuitry to power the entire sensor assembly module 200. In an embodiment, power module 240 may comprise circuitry, voltage regulators, capacitor banks, batteries, filters, amplifiers, wireless charging circuitry, and connectors. In an embodiment, power module 240 may comprise an internal battery that may be charged wirelessly or via an external power source. For example, an internal battery may be charged via USB as is well known in the art.

Communication module 230 may comprise an input-output (I/O) module 250. Communication module 230 may wirelessly communicate, and/or have a wired transceiver (communicate via wire). In an embodiment, communication module 230 may include a Bluetooth® capable transceiver, a WiFi capable transceiver, or any wireless capable transceiver as is well known in the art. Communication module 230 may comprise circuitry, RF antennas, modems, mixers, amplifiers, filters, radios, encoders, decoders, coders, modulators, converters, demodulators, baseband processors, USB drivers, connectors, or any components or circuitry required for wireless or wired communication as is well understood in the art. The communication module 230 may send and receive information to a second device 610. The information may be raw data or processed data. In an embodiment, the information sent or received may be encrypted. The communication module 230 may send and receive information to a smart device or external server (FIG. 6, 610), for example, via a wired connection utilizing the I/O module 250. For example, I/O module 250 may comprise a USB connector, or wirelessly.

Sensor module 210 may comprise one or a plurality of sensors. Sensor module 210 may comprise one, or more than one, of the following sensors: motion, vibration, sound, microphone, acceleration, angle, audio, biometrics, physiological, respiration, displacement, electric current, gyroscopic, infrared, heart, level, linear acceleration, breathing, orientation, or piezoelectric sensor. In an embodiment, sensor module 210 may comprise sensors that may help monitor for heart rate, breathing rate, and user movement. Sensor module 210 may send data to the signal condition module 215 or data to the processor module 220.

In an embodiment, sensory assembly module 200 may comprise an indication module 260. Indication module may be used to provide user feedback or mediation prompts. Indication module 260 may comprise one or more than one of the following: LEDs, audio, audio output, braille reader/writers, speakers, buzzer, sound chip, display, monitor, or vibrator. Indication module 260 may include circuitry, amplifier/s, filter/s, drivers, switches, and DACs or any circuitry as is well known in the art for providing indications with LEDs, audio, braille reader/writers, speakers, buzzer, sound chip, display, monitor, or vibrators.

Memory module 225 may comprise one or more memories. For example, memory module 225 may comprise RAM, ROM, Flash, or any combinations thereof. The memory may be included on one single “chip” or multiple “chips” as is well understood in the art. In an embodiment, the memory may be part of a processor chip. In an embodiment, Memory module 225 may comprise other forms of storage as is available in the art now or in the future.

The signal conditioning module 215 may, comprise amplifier/s, filter/s, and ADCs. Signal conditioning module 215 may receive information from the sensor module 210 and may condition the signals to forward on to processor module 220. In an embodiment, sensor module 210 may send information directly to the processor module 220. Signal conditioning module 210 may include a low pass filter to help filter out unwanted signals. Signal conditioning module 210 may amplify and convert analog signals from the sensor module into digital signals before sending them onto the processor module.

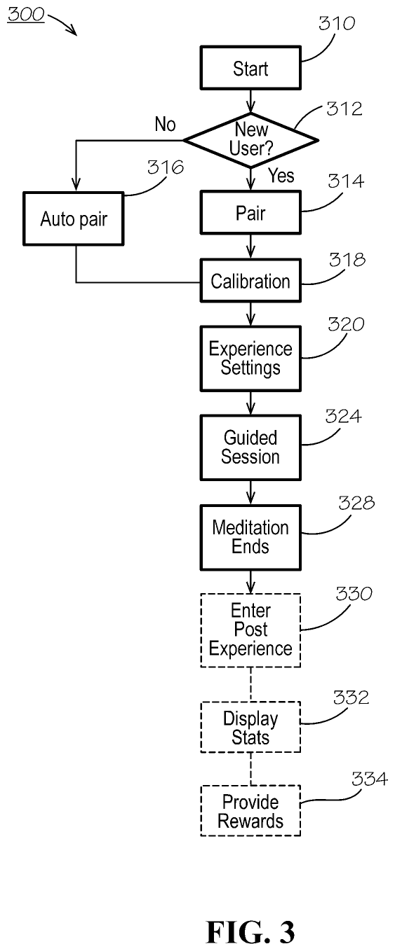

FIG. 3 illustrates a method 300 for meditation in an embodiment. The method may be used by a mediation software application. The method 300 provides improvements in the field of mediation. For example, unlike other devices that monitor brain patterns, the biometrics monitored herein are focused more on the breathing, heart, and movement of the user. This may allow the user to have thoughts without triggering inaccurate brain monitoring feedback or forcing the user to control their thoughts more than they desire. In other words, the user can think more freely and concentrate on their breathing. Moreover, most users can't objectively measure and compare their heart rates and breathing rates while meditating, nor mentally keep track of mediation data (statics) over time. Also, the group embodiment may not even be possible without the described embodiments. In addition, the methods, systems, and apparatus disclosed enable convenience and portability of the technology. For example, a user may use these improvements outside in nature: they do not need to be tied to a power supply, indoors, or bulky equipment. They do not have to wear any instruments, which may be uncomfortable. These, amongst other reasons, make mediation more efficient, objective, social, and relaxing, thus, improving the field.

At step 310 starting a mediation session; the application may start a meditation session. At step 312, determining if the user is new; if the user is new, the process then goes to the pairing and/or registration step at 314. At step 314 pairing; after the new user registers, then the sitting apparatus and the second device may pair at step 314. If the user isn't new at step 312, then the process goes onto the auto pairing; auto pair step 316. The pairing of Bluetooth (or other protocols) devices is well known in the art. In an embodiment, the sitting apparatus may be paired with the second device via a sitting apparatus identifier. For example, the sitting apparatus may come with a barcode or unique code. The second device may then scan the bar code or have the code entered to pair. At step 318 calibrating; after pairing, the process may then go onto the calibration step 318. In an embodiment, factory calibration of the sitting apparatus may occur once prior to shipping and calibration of each session may occur prior to each meditation session. Calibration per session may be preformed to correct for session variations, for example, a user may be sitting slightly off the sensor module or wearing extra clothing, thus, the calibration step 318 may help optimize the meditation sessions. At step 320 prompting for experience settings; at step 320, the user may be prompted for their meditation experience setting/s. In an embodiment, the user may enter what mood they are in (pre-meditation state). For example, the user may select a pre-mediation state as anxious, depressed, happy, neutral, or calm. Selecting a pre-meditation state may help the meditation software application keep track of useful statistics in order to aid the user in future states of mediation. In an embodiment, a user may indicate what type of meditation feedback they desire. For example, they may indicate they want audio feedback, tactile feedback, visual feedback, a guided session mode, silent mode, or a group session, or any combinations thereof. At step 324 beginning guided session; at step 324 the guided meditation session may begin. It may begin by user prompt/input (e.g. push/touch button) or automatically (e.g. pre-timed auto start) based on user preferences. In an embodiment, voice commands and voice prompts may be enabled. For example, a user may speak the command to “start” or “stop” the meditation processes. The sitting apparatus or the second device (controlled by the mediation software application) may issue and receive voice commands/prompts. At step 328 ending mediation session; at step 328, the meditation session's ending is determined and ends. Meditation may end based on user input e.g. push/touch button, or automatically (e.g. pre-timed stop watch timer or a goal that has been reached, or based on user preferences. Optionally, at step 330, entering post experiences; a user may enter their post meditation mood for session statistics. For example, the user may indicate that their post mood is relaxed, calm, neutral, or anxious. Optionally, at step 332, displaying statistics; the user's current and/or historical statistics (e.g. session history) may be displayed. In an embodiment, the post experiences (as well as real-time feedback or statistics) provided to a user may include, but are not limited to, a stillness score. The stillness score may be a number from zero to ten with ten being the highest. The time that the user stayed in a mediation state may be provided. Also, the times of each mediation state for Alpha, Theta, and Delta (see below) may be provided. A single calmness trace may be displayed that indicates the user's Alpha, Theta, and Delta states over time. The time it took to get into a state may be provided as well as the time the user stayed in a particular state. The starting heart rates and breathing rates as well as their end rates may be provided. The lowest heart and breathing rate may also be provided. The coherence between heart and breathing rate may also be included. The user's heart rate variability and breathing pattern data may be provided. In an embodiment, the post experiences (as well as real-time feedback or statistics) provided may be graphically displayed to the user. For example, the breathing or heart rate patterns may be displayed as colored traces. The post experiences (as well as real-time feedback or statistics) may also be provided in a numerical expression as well as acoustically expressed or any combinations thereof. In an embodiment, the user may setup their personally desired mode of post-experience (as well as real-time feedback or statistics) feedback presences. In an embodiment, different traces of the feedback may converge onto a single trace to show mediation state achievement and diverge to show non-mediation states. And finally, optionally, at step 334, providing rewards; rewards or badges based on the user's performance may be provided.

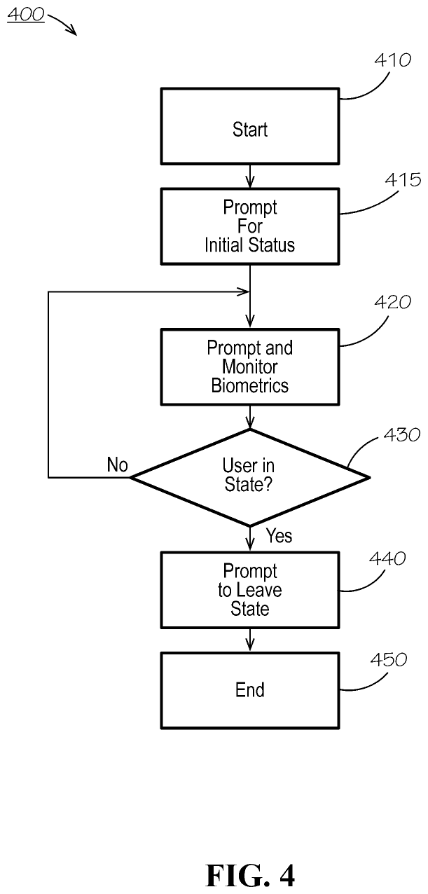

FIG. 4 illustrates a guided mediation method 400 in an embodiment. The method may be used by a meditation software application. In an embodiment. At step 410, beginning mediation; indication that the meditation has begun may be provided to the user. At step 415, prompting for initial status; the user may be prompted to analyze their initial thoughts and emotions (pre-meditation state or initial status). At step 420, prompting user and monitoring biometrics; the user's biometrics may be monitored and the user may be prompted to achieve specific goals. Examples of the type of biometrics that may be measured, obtained, or monitored may include ballistocardiography, heart rate, breathing rate, and movement, The user may prompted to begin breathing exercises. For example, the user may be told to “relax more,” “breath slower”, “breath deeper,” “stay still,” “breath in” or “breath out,” etc. The user may have audio, visual, tactile, or any combination thereof for prompting through the breathing exercises. The user may be prompted to slow down their breathing rate, which in turn, may slow down their heart rate. The user may be prompted to try and make their breathing rate consistent. In an embodiment, a user may be prompted to reach an Alpha state. The Alphas state may prompt a user to inadvertently drop their heart rate to between 3-5%. A user may be prompted to reach a Theta state. A Theta state may prompt the user in addition to dropping their heart rate by 3-5%, to also drop their breathing rate by 3-5%. A user may be prompted to reach a Delta state. A delta state may prompt a user in addition to dropping their heart rate by 3-5% and their breath rate by 3-5%, to also have a breath rate that remains consistent for a minimum of between 2-3 minutes. The various states may have different labels, e.g. “level one,” “level, two,” “blue,” “green,” etc. It is the inventors' intent that a variety (numerous) labeling of the states and number of the states may be used and are contemplated to be included herein. In an embodiment, the state the user is in may be dynamically displayed as a single calmness trace.

Also, the meditation software application may direct the sitting apparatus to measure the user's biometrics. The sitting apparatus in conjunction with the meditation software application may scan the user's heart rate (e.g. body scan), breathing rate, and rhythm/synchronization of the two rates together (e.g. induction), and provide specific custom guidance to help the user reach a target meditation state. A user may be directed to breath slower, relax more, be stiller, or breath deeper. Once a user has reached a meditation state, at step 430, determining if user is in state; the user may be monitored to see if they stay within the reached state or not. If the user drifts out of the state, then the process may go back to step 420 and the user may be guided back into a meditation state. Finally, once the user is finished with their meditation session, at step 440, prompting to leave state; the user may be guided back into an active or alert state. In an embodiment, a user may be guided back to an alert state by way of commands or prompts. For example, they may be provided voice commands and prompts based on the sensor information analysis. For example, the user may be told to “come back into you body and mind,” “be mindful of your surroundings,” or “take note of your thoughts and feelings at the end of this meditation”. The prompting maybe visual, audio, tactile, or any combinations thereof. For example, a “beep” or vibration may indicate a direction for the user. At step 450, ending; the process ends at step 450.

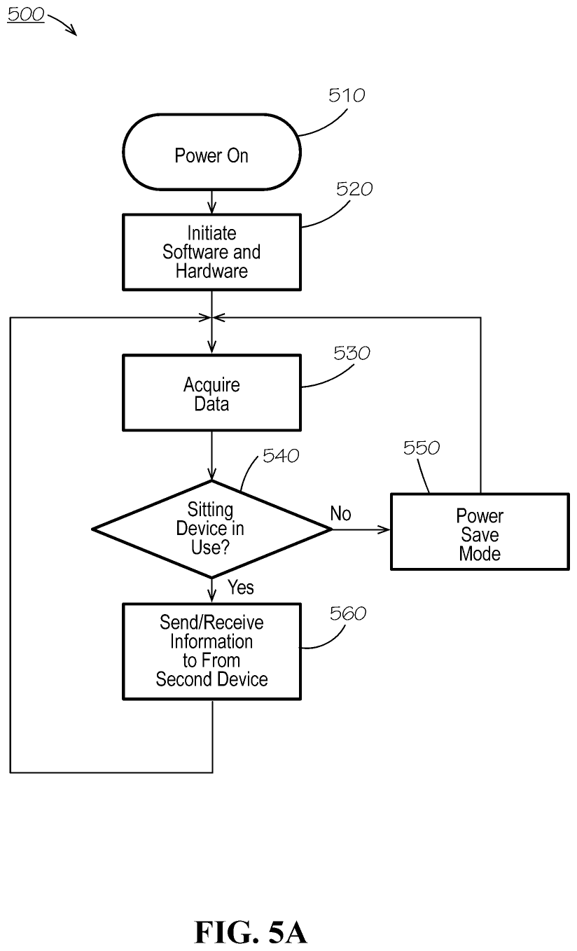

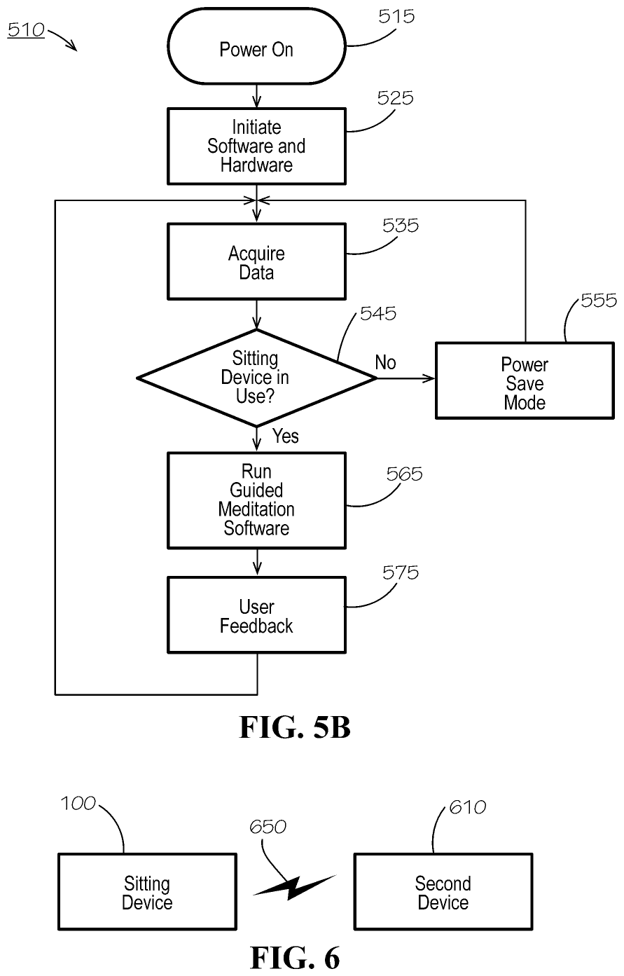

FIG. 5A illustrates a method 500 for the sitting apparatus in an embodiment. At step 510, powering on; the sitting apparatus (e.g. 100) may be turned on (power on). The device may be turned on via a physical power on button, by a sensor that detects the sitting apparatus will be in use, or a virtual power on button (a touch screen icon on the second device). At step 520, initializing; the sitting apparatus' software and hardware may be initialized. At step 530, obtaining data; the sitting apparatus may acquire user information or data. E.g. sensor data either raw or processed. At step 540, determining if in use; the sitting apparatus may determine if the device is in use or not. If the device is in use, then the process may go on to step 560. If the device isn't in use, then the process may go on to step 550, entering power saving mode; power save mode and back to the acquiring information/data step 530. At step 560, sending and receiving information; the sitting apparatus may send and receive information to and from a second device. Once the device is detected to be no longer in use the process may end (or be turned off).

FIG. 5B illustrates a method 510 for the sitting apparatus in another embodiment. At step 515, powering on; the sitting apparatus (e.g. 100) may be turned on (power on). At step 525, initializing; the sitting apparatus' software and hardware may be initialized. At step 535, obtaining data; the sitting apparatus may acquire user information or data. E.g. sensor data either raw or processed. At step 545, determining if in use; the sitting apparatus may determine if the device is in use or not. If the device is in use, then the process may go on to step 565. If the device isn't in use, then the process may go on to step 555, entering power saving mode; power save mode and back to the acquiring information/data step 535 At step 565, beginning guided session; the sitting apparatus may run its guided mediation software. For example, the methods of FIG. 3 and FIG. 4 may be used. At step 575, user feedback may be provided. The feedback may utilize the indication module 260 and may be audio, tactile, visual, or any combination thereof. Once the device is detected to be no longer in use, or is turned off, the process may power off and end.

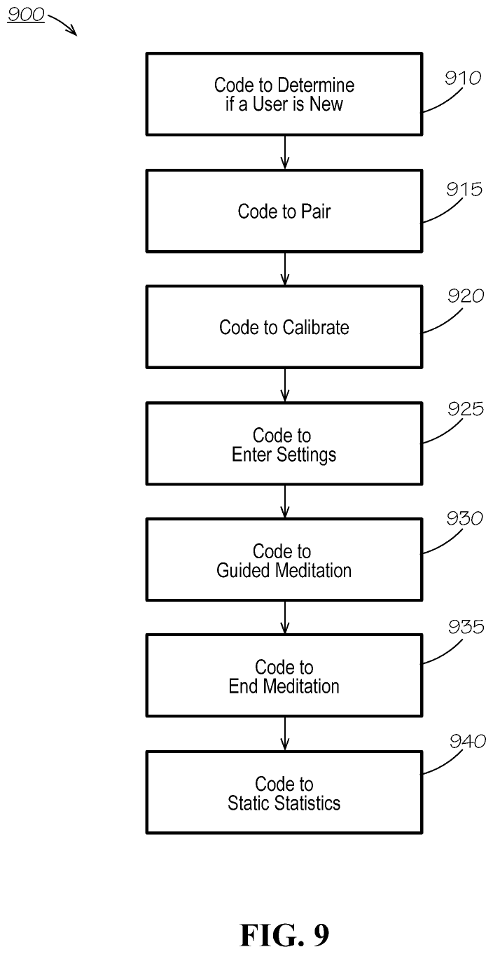

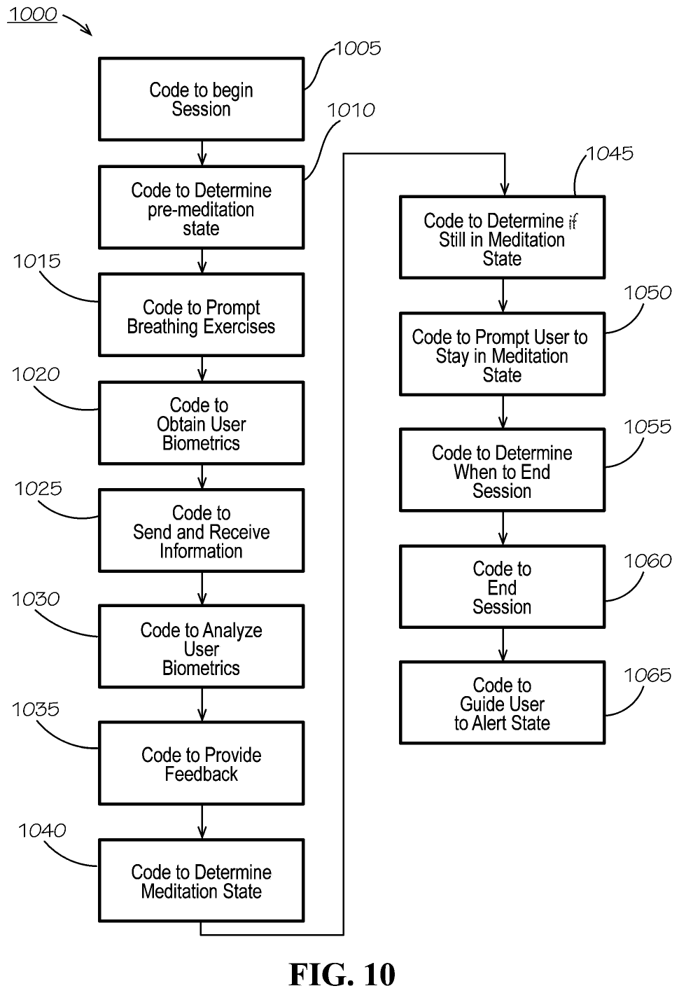

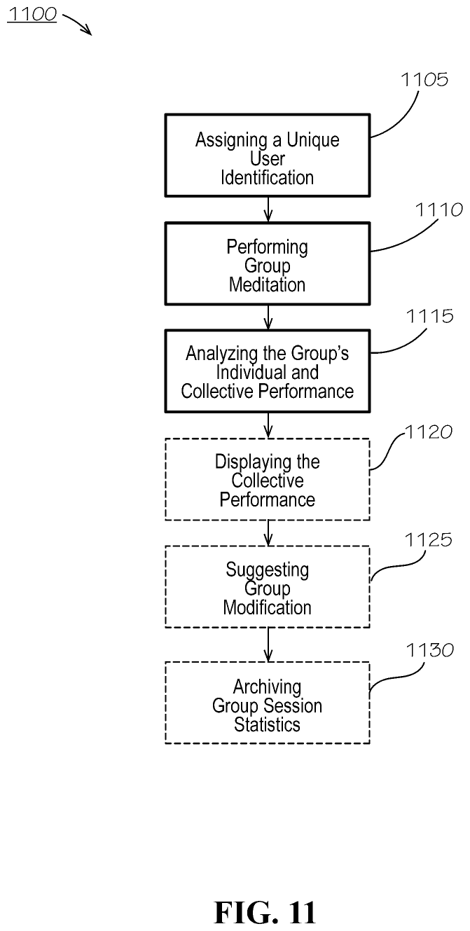

FIG. 6 illustrates a system 600 for mediation in an embodiment. Sitting apparatus (or sitting device) 100 may communicate wirelessly or wired 650 with a second device 610. Sitting apparatus 100 may be any of the described sitting apparatus and method embodiments and the second device may be any of the describe second devices and methods. For example, second device 610 may use the methods or codes as described in FIG. 3, FIG. 9, FIG. 10, or FIG. 11 etc. In an embodiment, sitting apparatus 100 may use the method of FIG. 5A.

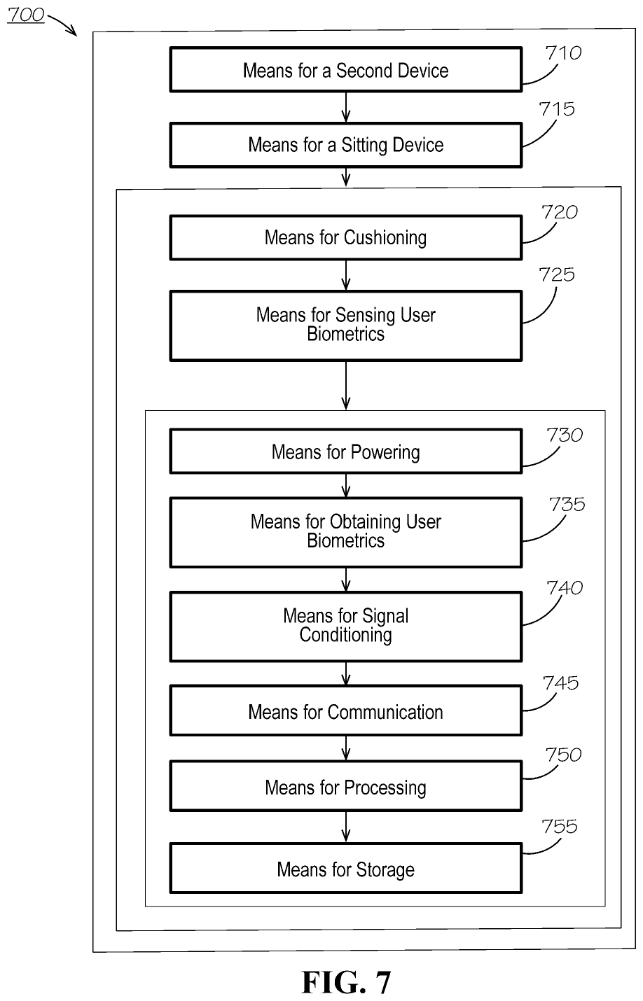

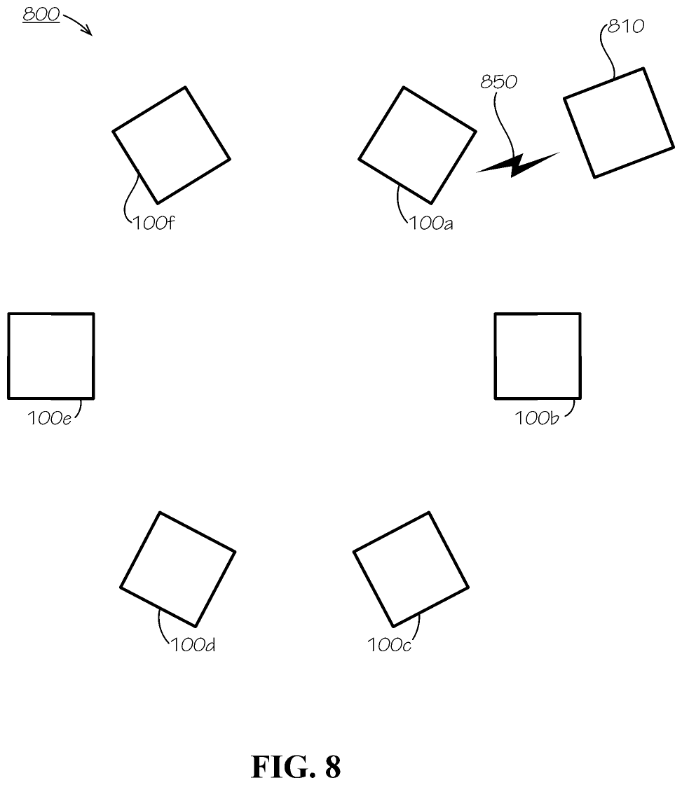

FIG. 7 illustrates a system 700 in an embodiment. The system 700 may include means for a second device 710, the second device may have a meditation software application. System 700 may have means for a sitting apparatus (or sitting device) 715 for meditation. The sitting apparatus 715 may have means for cushioning 720, means for sensing user biometrics 725: the means for sensing user biometrics may include means for powering the sitting apparatus 730, means for obtaining user biometrics 735, means for signal conditioning 740, means for communicating 745, the means for communicating may have means for communicating with the second device meditation software application, means for processing 750, the means for processing may have means for sending and receiving information to the second device, and means for storage 755.

FIG. 8 illustrates a group meditation system 800 in an embodiment. In an embodiment, sitting apparatuses (or sitting devices) 100a-f may be in communication 850 (wirelessly or wired) with a second device 810. Any number of sitting apparatuses may be in the group as long as there are at least two sitting apparatuses to form a group. Sitting apparatuses 100a-f may be or utilize any of the described sitting embodiments and methods and second device 810 may be any of the described second devices, or utilize any of the described codes or methods. In an embodiment, sitting apparatus 100 may use the method of FIG. 5A or 5.B. In an embodiment, system 800 may use the method of FIG. 11. The system 800 may control and monitor a group session: how well the group synchronizes their meditation states and breathing together may be monitored, guided, and historically tracked.

FIG. 9 illustrates code for meditation 900 in an embodiment. At 910 there may be code to determine whether a user is new: if a user is new, then at 915 there may be code to prompt a user to register and pair, if a user is not new, then at 915 there may be code to automatically pair. At 920, there may be code to calibrate a meditation session. At 925, there may be code to allow a user to enter meditation settings. At 925, in addition, there may be code to allow a user to enter a pre-meditation state. A state of anxious, depressed, happy, neutral, and calm may be entered. Also, there may be code for allowing a user to select feedback settings. Feedback settings may include audio feedback, a guided session mode, silent mode, visual, tactile, or and group session. At 930, there may be code to begin a guided meditation. At 935, there may be code to determine an end to the mediation session and code to end the guided meditation session. And at 940, there may be code to store the guided meditation session statistics. There may be code for code 900 to also optionally include code to prompt user for a mood after the guided meditation ends, code to display statistics, and code to present rewards.

FIG. 10 illustrates code for guided meditation 1000 in an embodiment. At 1005, there may be code to begin a mediation session. At 1010, there may be code to prompt a user for pre-mediation state or determine a user's pre-mediation state. At 1015, there may be code to prompt user to begin breathing exercises. At 1020, there may be code to instruct a meditation sitting apparatus to obtain or measure a user biometric information. At 1025, there may be code to send and receive information, code to transmit user biometrics and code to receive the user biometric information. At 1030, there may be code to analyze the user biometric information. At 1035, there may be code to provide feedback to user. At 1040, there may be code to determine when user reaches a meditation state. At 1045, there may be code to monitor (or determine) if user stays within the meditation state. If the user drifts out of the meditation state, then at 1050 there may be code to return to the code to prompt the user to stay within the mediation state. For example, the user may be prompted to perform begin breathing exercises again 1015. If user stays in a mediation state, then at 1055 there may be code to determine when to end the meditation session end. At 1060 there may be code to end the meditation. And at 1065 there may be code to guide a user back into an active or alert state.

FIG. 11 illustrates a method for group mediation 1100 in an embodiment. At step 1105, assigning a unique identifier to each user in the group may be done. Each user in the group may be assigned a unique identifier that allows them to be identified during feedback or statics. For example, when displaying feedback, the users may be assigned a color to illustrate their unique identifier. For example, the traces may be colored and/or patterned to represent each user in the group session. At step 1110, performing group mediation may be done. Individuals in the group may be monitored and guided per the disclosed embodiments. In addition, the group as a whole may also be monitored and guided as a whole. At step 1115, analyzing the group's individual and collective patterns may be done. For example, rather than an individual's convergence of breathing rates, the entire groups breathing traces may be displayed to converge as a unit (group). Optionally, at step 1120, displaying the collective patterns may be done. Optionally, at step 1125, suggesting group modification may be done. For example, users in the group may be prompted to reach group goals, or the group may be prompted as a whole. Optionally, at step 1130, archiving group session statics may be done. The process ends when the group session is concluded.

In other embodiments, there may be a sitting apparatus for meditation, comprising: a cushion; a sensor assembly module, the sensor assembly module comprising: a power module adapted to power the sensor assembly module; an input-output module; an indication module; a communication module; a signal conditioning module; a sensor module adapted to obtain user biometrics; a processor module, the processor module comprising a meditation software application; and a memory module coupled to the processor module. The sitting apparatus further comprising: a removably secure cover. The sitting apparatus further comprising: a second cushion; and the sensor assembly module is in-between the cushion and the second cushion. The sitting apparatus, wherein the sensor module comprising a piezoelectric sensor. The sitting apparatus wherein the sensor module measures heart rate, breathing rate, and movement. The sitting apparatus wherein the indication module comprises a display, a LED, an audio output, a braille writer, a speaker, a buzzer, a sound chip, a monitor, and vibrator. The sitting apparatus wherein the sensor module comprises an outer housing. The sitting apparatus wherein the communication module comprises an input-output module comprising USB capability. The sitting apparatus wherein the processor module adapted to detect if the sitting apparatus is in use and enter a power on state; and adapted to detect if the sitting apparatus is not in use and enter a power down state. The sitting apparatus wherein the meditation software application comprises: code to calibrate a meditation session; code to allow a user to enter meditation settings; code to begin guided meditation; code to end guided meditation; and code to store the guided meditation session statistics. The sitting apparatus wherein the code to begin guided meditation, comprises: code to prompt user for pre-mediation state; code to prompt user to begin breathing exercises; code to instruct a meditation sitting apparatus to obtain user biometric information, code to process the user biometric information; code to analyze the user biometric information; code to provide feedback to user; code to determine when user reaches a meditation state; code to monitor if user stays within the meditation state, if user drifts out of the meditation state, then code to return to the code to prompt user to begin breathing exercises, if user stays in mediation state, then code to determine meditation session end; code to end meditation; and code to guide user back into an active or alert state.

In other embodiments, there may be a sitting apparatus for meditation, comprising: means for cushioning; means for sensing user biometrics, the means for sensing user biometrics comprising: means for powering the sitting apparatus; means for obtaining user biometrics means for signal conditioning; means for communicating, the means for communicating with a second device meditation software application; means for processing, the means for processing comprising means for sending and receiving information to the means for the second device; and means for storage.

In another embodiment, there may be a method for group mediation, comprising: assigning a unique user identification; performing group mediation; analyzing the group's individual and collective performance; and displaying the collective performance. The method further comprising: suggesting group modifications. The method further comprising, archiving group session statistics.

In another embodiment, there may be a method for mediation, comprising: determining whether a user is new: if a user is new, then prompting a user to register and pair, if a user is not new, then automatically pairing; calibrating a meditation session; allowing a user to enter meditation settings; beginning guided meditation; ending guided meditation; and storing the guided meditation session statistics. The method further comprising: prompting a user for a mood after the guided meditation ends. The method further comprising: displaying statistics. The method, further comprising: presenting rewards. The method, wherein the meditation settings comprises: allowing a user to enter a pre-meditation state; and allowing a user to select feedback preferences. The method, wherein allowing a user to enter a pre-meditation state is one of anxious, depressed, happy, neutral, and calm; and wherein the allowing a user to select feedback settings is an audio feedback, a guided session mode, a silent mode, a visual, a tactile, a group session, or any combinations thereof. The method, wherein, beginning guided meditation comprises: prompting a user for pre-mediation state; prompting user to begin breathing exercises; instructing a meditation sitting apparatus to obtain and transmit user biometric information, receiving the user biometric information; analyzing the user biometric information; providing feedback to user; determining when user reaches a meditation state; monitoring if user stays within the meditation state, if user drifts out of the meditation state, then returning to the prompting breathing exercises, if user stays in mediation state, then determining meditation end; ending meditation; and guiding user back into an alert state.

In other embodiments, the processing modules may be implemented using a shared processing device, individual processing devices, or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions.

The described embodiments or any part(s) or function(s) thereof, may be implemented using hardware, software, or a combination thereof, and may be implemented in one or more computer systems or other processing systems. A computer system for performing the operations of the described embodiments and capable of carrying out the functionality described herein can include one or more processors connected to a communications infrastructure (e.g., a communications bus, a cross-over bar, or a network). Various software embodiments are described in terms of such an exemplary computer system. After reading this description, it will become apparent to a person skilled in the relevant art(s) how to implement the embodiments using other computer systems and/or architectures.

The foregoing description of the preferred embodiments has been presented for purposes of illustration and description. It is not intended to be exhaustive or to limit the embodiments to the precise form or to exemplary embodiments disclosed. Obviously, many modifications and variations will be apparent to practitioners skilled in this art. Similarly, any process steps described might be interchangeable with other steps in order to achieve the same result. The embodiments were chosen and described in order to best explain the principles of the embodiments and its best mode practical application, thereby to enable others skilled in the art to understand the various embodiments and with various modifications as are suited to the particular use or implementation contemplated. It is intended that the scope of the embodiments be defined by the claims appended hereto and their equivalents. Reference to an element in the singular is not intended to mean “one and only one” unless explicitly so stated, but rather means “one or more.” Moreover, no element, component, nor method step in the described disclosure is intended to be dedicated to the public regardless of whether the element, component, or method step is explicitly recited in the following claims. No claim element herein is to be construed under the provisions of 35 U.S.C. Sec. 112, sixth paragraph, unless the element is expressly recited using the phrase “means for . . . .”

In addition, the conjunction “and” when used in the claims is meant to be interpreted as follows: “X, Y and Z” means it can be either X, Y or Z individually, or it can be both X and Y together, both X and Z together, both Y and Z together, or all of X, Y, and Z together.

It should be understood that the figures illustrated in the attachments, which highlight the functionality and advantages of the described embodiments, are presented for example purposes only. The architecture of the described embodiments are sufficiently flexible and configurable, such that it may be utilized (and navigated) in ways other than that shown in the accompanying figures.

Furthermore, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the described embodiments in any way. It is also to be understood that the steps and processes recited in the claims need not be performed in the order presented.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function. A process or method may be implemented with a processor, or similar device, or any combination of hardware and software.

Moreover, a storage medium may represent one or more devices for storing data, including read-only memory (ROM), random access memory (RAM), magnetic disk storage mediums, optical storage mediums, flash memory devices and/or other machine-readable mediums, processor-readable mediums, and/or computer-readable mediums for storing information. The terms “machine-readable medium”, “computer-readable medium”, and/or “processor-readable medium” may include, but are not limited to non-transitory mediums such as portable or fixed storage devices, optical storage devices, and various other mediums capable of storing, containing or carrying instruction(s) and/or data. Thus, the various methods described herein may be fully or partially implemented by instructions and/or data that may be stored in a “machine-readable medium”, “computer-readable medium”, and/or “processor-readable medium” and executed by one or more processors, machines and/or devices. Moreover, a micro processor, or similar device may have internal or external memory associated with it.

The various features of the embodiments described herein can be implemented in different systems without departing from the embodiments. It should be noted that the foregoing embodiments are merely examples and are not to be construed as limiting the embodiments. The description of the embodiments is intended to be illustrative, and not to limit the scope of the claims. As such, the described teachings can be readily applied to other types of apparatuses and many alternatives, modifications, and variations will be apparent to those skilled in the art.

What is claimed is:

1. A sitting apparatus for meditation, comprising:
a cushion;
a sensor assembly module, the sensor assembly module comprising:
a power module adapted to power the sensor assembly module;
a sensor module adapted to obtain user biometrics;
a signal conditioning module adapted to condition signals from the sensor module;
a communication module;
a processor module, the processor module adapted to send and receive information to a second device by utilizing the communication module, to send user biometric information to the second device meditation software application; and
a memory module coupled to the processor module.

2. The sitting apparatus of claim 1, further comprising:
a removably secure cover.

3. The sitting apparatus of claim 1, further comprising:
a second cushion; and
the sensor assembly module is in-between the cushion and the second cushion.

4. The sitting apparatus of claim 1, wherein the sensor module comprising a piezoelectric sensor.

5. The sitting apparatus of claim 1, wherein the sensor module obtains user heart rate, breathing rate, and movement information.

6. The sitting apparatus of claim 1, wherein the communication module further comprising: an input-output module comprising USB capability.

7. The sitting apparatus of claim 1, wherein the processor module adapted to pair the sitting apparatus with the second device, to calibrate a meditation session, to receive control information from the second device, and to send user biometric information to the second device.

8. The sitting apparatus of claim 1, wherein the sensor module comprises an outer housing.

9. The sitting apparatus of claim 1, wherein the signal conditioning module filters unwanted noise.

10. The sitting apparatus of claim 1, wherein the processor module adapted to detect if the sitting apparatus is in use and enter a power on state; and
adapted to detect if the sitting apparatus is not in use and enter a power down state.

11. A non-transitory computer readable medium encoded with processing instructions, for meditation software application, comprising:
code to determine whether a user is new: if a user is new, then code to prompt a user to register and pair, if a user is not new, then code to automatically pair;
code to calibrate a meditation session;
code to allow a user to enter meditation settings;
code to begin guided meditation;
code to end guided meditation; and
code to store the guided meditation session statistics.

12. The non-transitory computer readable medium of claim 11, further comprising:
code to prompt user for a mood after the guided meditation ends.

13. The non-transitory computer readable medium of claim 11, further comprising:
code to display statistics.

14. The non-transitory computer readable medium of claim 11, further comprising:
code to present rewards.

15. The non-transitory computer readable medium of claim 11, wherein the code to allow a user to enter meditation settings comprises:
code to allow a user to enter a pre-meditation state; and
code for allowing a user to select feedback preferences.

16. The non-transitory computer readable medium of claim 15, wherein the code to allow a user to enter a pre-meditation state is one of anxious, depressed, happy, neutral, and calm; and
wherein the code for allowing a user to select feedback settings is an audio feedback, a guided session mode, a silent mode, a visual, a tactile, a group session, or any combinations thereof.

17. The non-transitory computer readable medium of claim 11, wherein, the code to begin guided meditation comprises:
code to prompt user for pre-mediation state;
code to prompt user to begin breathing exercises;
code to instruct a meditation sitting apparatus to obtain and transmit user biometric information, code to receive the user biometric information;
code to analyze the user biometric information;
code to provide feedback to user;
code to determine when user reaches a meditation state;
code to monitor if user stays within the meditation state, if user drifts out of the meditation state, then code to return to the code for prompting breathing exercises, if user stays in mediation state, then code to determine meditation end;
code to end meditation; and
code to guide user back into an alert state.

18. A system for mediation, comprising:
means for a second device, the second device being capable of running instructions for a meditation software application;
means for a sitting apparatus, the sitting apparatus being capable of use with the second device for meditation, comprising:
means for cushioning;
means for sensing user biometrics, the means for sensing user biometrics comprising:
means for powering the sitting apparatus;
means for obtaining user biometrics;
means for signal conditioning;
means for communicating, the means for communicating having means for communicating with the second device meditation software application;
means for processing, the means for processing comprising means for sending and receiving information to the means for the second device; and
means for storage.

19. The system of claim 18, wherein the instructions for a meditation software application comprises:
code to determine whether a user is new: if a user is new, then code to prompt a user to register and pair, if a user is not new, then code to automatically pair;
code to calibrate a meditation session;
code to allow a user to enter meditation settings;
code to begin guided meditation;
code to end guided meditation; and
code to store the guided meditation session statistics.

20. The system of claim 19, wherein the code to begin guided meditation comprises:
code to prompt user for pre-mediation state;
code to prompt user to begin breathing exercises;
code to instruct a meditation sitting apparatus to obtain and transmit user biometric information, code to receive the user biometric information;
code to analyze the user biometric information;
code to provide feedback to user;
code to determine when user reaches a meditation state;
code to monitor if user stays within the meditation state, if user drifts out of the meditation state, then code to return to the code to prompt user to begin breathing exercises, if user stays in mediation state, then code to determine meditation session end;
code to end meditation; and
code to guide user back into an active or alert state.
